(19) United States
(12) Patent Application Publication
(10) Pub. No.: US 2007/0086983 A1
(43) Pub. Date: Apr. 19, 2007

(54) Endothelium-targeting nanoparticle for reversing endothelial dysfunction

(75) Inventor: Cynthia J. Meininger, Georgetown, TX (US)

Correspondence Address: MEDLEN & CARROLL, LLP, Suite 350, 101 Howard Street, San Francisco, CA, 94105, US

(73) Assignee: The Texas A&M University System

(21) Appl. No.: 11/545,370

(22) Filed: Oct. 10, 2006

Related U.S. Application Data
(63) Continuation of application No. 10/934,226, filed on Sep. 3, 2004, now abandoned.
(60) Provisional application No. 60/481,336, filed on Sep. 5, 2003.

(51) Int. Cl. A61K 48/00

(52) U.S. Cl. 424/93.2; 514/44

(57) ABSTRACT

The present invention includes delivery of isolated and purified nucleic acids that encode GTPCH proteins in nanoparticles for the treatment of endothelial cells damaged by diabetes, smoking, dyslipidemia, hypertension, and cardiovascular disease. The nanoparticles contain a nucleic acid sequence, polymer and a targeting ligand. The targeting ligand facilitates the selective delivery of the nucleic acid sequence to damaged endothelial cells. Examples involving a nucleic acid sequence encoding GTP-cyclohydrolase I (GTPCH), PEG/PEI polymers, and a monoclonal antibody or other molecule that binds to the lectin-like oxidized low density lipoprotein (LDL) receptor-1 (Lox-1) or associated molecules are presented.

This application claims priority to U.S. Provisional Patent Application Ser. No. 60/481,336, filed Sep. 5, 2003. Without limiting the scope of the invention, its background is described in connection with diabetes, hypertension, dyslipidemia and smoking.

### TECHNICAL FIELD OF THE INVENTION

The present invention relates to compositions and methods for the delivery of nucleic acids to endothelial cells and, more specifically, to nanoparticle-mediated delivery of nucleic acids to damaged blood vessels in individuals with cardiovascular disease resulting from diabetes, hypertension, dyslipidemia, and/or smoking.

### DESCRIPTION OF RELATED ART

Diabetes is one of the most prevalent and costly chronic diseases in the U.S. According to the Centers for Disease Control and Prevention, one in three Americans born in the year 2000 will develop diabetes. The prevalence is even higher for Hispanics where the estimated lifetime risk is 45% for males and 53% for females. One in ten health care dollars in the U.S. is spent on diabetes and most of this is for treatment of vascular complications of the disease. Endothelial cell dysfunction is a major cause of these complications. As such, there has been much recent interest in developing strategies to reverse or retard endothelial dysfunction in order to modify the natural history of diabetic vascular disease.

Targeting genes to specific blood vessels damaged by disease offers therapeutic promise for reversing that damage and preventing vascular complications associated with the disease. Viral vectors, liposomes and naked DNA have been used for delivery of therapeutic genes to vascular tissues, but none of these approaches are specific for dysfunctional endothelial cells.

### SUMMARY OF THE INVENTION

The present inventor has recognized that there exists a need for improved delivery systems capable of delivering genes that will ameliorate the effects of vascular disease. The delivery system should overcome the shortcomings of the previously reported research by using selective delivery to endothelial cells damaged by vascular disease. Currently available delivery systems, e.g., viral vectors, fail to provide the required expression levels, specificity of localization and have caused some safety concerns for use in humans. As such, a need exists for an endothelial cell-specific delivery system that overcomes the cellular dysfunction associated with decreased production of essential cofactors and/or precursors for the enzyme nitric oxide synthase.

The present invention includes a polymerized nanoparticle with a targeting ligand that is prepared and used to deliver an isolated and purified nucleic acid sequence encoding a GTP cyclohydrolase (GTPCH) polypeptide to damaged endothelial cells. The delivery of the GTPCH nucleic acid promotes long-term production of the GTPCH protein in endothelial cells of individuals with either type I (insulin-dependent) or the more prevalent type II (insulin-resistant) diabetes. The delivery system can be used to treat endothelial damage caused by diabetes, smoking, dyslipidemia, hypertension, and/or cardiovascular disease.

The present invention includes materials and methods for the delivery of one or more nucleic acids to cells of a recipient subject. Briefly, the method includes contacting Lox-1-expressing endothelial cells with a delivery system, the delivery system including: a ligand associated with a carrier capable of binding to Lox-1-expressing endothelial cells and an isolated and purified nucleic acid associated with the carrier encoding a GTP-cyclohydrolase I and administering the delivery system to the recipient subject under conditions such that at least a portion of the Lox-1-expressing endothelial cells are contacted by the delivery system. The cells may be part of a vascular tissue, for example, Lox-1-expressing endothelial cells that are cells damaged by or reactive to a disease, e.g., diabetes, dyslipidemia, hypertension and/or cardiovascular disease. The nucleic acid may be found within an expression vector, which may include a promoter sequence operably linked to the nucleic acid, e.g., a promoter sequence that is a viral promoter sequence. The ligand associated with the carrier may be an antibody reactive with Lox-1, an antigen binding portion of an antibody, an antigen binding portion of a monoclonal antibody or other peptide. Examples of carriers for use with the present invention include: polymers, liposomes, LDL, modified LDL, a nanocore, a nanoparticle or a combination thereof. When used in a subject the present invention may be administered by intravenous injection and the subject may be a human. The method of the present invention may also include testing the recipient subject for evidence of increased nitric oxide synthesis.

In another embodiment, the present invention includes a delivery system having a Lox-1 binding agent and a nucleic acid encoding a GTP-cyclohydrolase I associated with a carrier, wherein the Lox-1 binding agent targets the delivery system to Lox-1 expressing cells for delivery of the nucleic acid. The carrier may be a polymer, a liposome, an LDL molecule, an oxidized or modified LDL molecule, a nanocore, a nanoparticle or a combination thereof. A method for delivering a nucleic acid to cells of a subject may also include providing a subject with Lox-1-expressing endothelial cells a nanoparticle, the nanoparticle having a targeting ligand for Lox-1 on endothelial cells and a nucleic acid encoding a GTP-cyclohydrolase I. For example, the administration of the delivery system to the subject will be under conditions such that at least a portion of the Lox-1-expressing endothelial cells are contacted by the delivery system. The contacted cells may be part of vascular tissue, e.g., vascular tissue damaged by or reactive to a disease or trauma, e.g., diabetes, dyslipidemia, hypertension and cardiovascular disease. In one embodiment, the targeting ligand is an antibody that is specific for Lox-1, which may be a polyclonal or monoclonal antibody, fragments thereof or even peptides that bind specifically to Lox-1 or other entities associated with Lox-1. The targeting ligand is incorporated into a nanoparticle, e.g., polymeric nanoparticle that may have a core and a polymeric surface, wherein the nucleic acid is associated with the core and the targeting ligand is associated with the surface. By way of example, the nanoparticle may be between about 50 and about 100 nanometers in size, however, they may be larger or smaller and have any shape.

Yet another example of the present invention is a delivery system that includes a targeting ligand that binds to Lox-1-expressing endothelial cells and a nucleic acid encoding a GTP cyclohydrolase I, wherein the ligand and the nucleic acid are associated with a nanoparticle. In this example, the targeting ligand may or may not bind to Lox-1 itself. For example, the targeting ligand may bind specifically to a Lox-1 associated protein or even to an oxidized LDL molecule attached to the Lox-1 protein in a “sandwich-type” binding. Generally, the ligand target will be associated with the cell surface.

Nanoparticles for use with the present invention include, e.g., a biocompatible polymer, a biodegradable polymer, a conductive polymer or combinations thereof. Examples of polymers for making the nanoparticles taught herein include poly(ethylene imine), poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, conductive polymers, and combinations thereof. The present invention may also include natural polymers comprising carboxymethyl cellulose, and hydroxyalkylated celluloses such as hydroxyethyl cellulose and methylhydroxypropyl cellulose, polypeptides, polysaccharides or carbohydrates, polysucrose, hyaluronic acid, dextran, heparan sulfate, chondroitin sulfate, heparin, and alginate, and proteins such as gelatin, collagen, albumin, and ovalbumin, other copolymers, and combinations thereof.

Yet another embodiment of the present invention is a method for expressing a polypeptide in an endothelial cell by providing a non-viral composition that specifically targets endothelial cells with a nucleic acid that encodes one or more genes for a polypeptide that reverses endothelial cell dysfunction caused by decreased intracellular tetrahydrobiopterin concentration. The polypeptide is expressed by delivering the composition to the cell under conditions that permit transfer of the composition into the cell and expression of the selected polypeptide. The invention also includes an amelioration of cellular dysfunction by providing a composition that specifically targets a dysfunctional endothelial cell with a targeting ligand that binds specifically to endothelial cells and delivers a nucleic acid the encodes one or more genes that increase intracellular tetrahydrobiopterin concentration under conditions that permit transfer of the composition into the cell. The targeting ligand may be, e.g., an antibody, an antibody fragment, a peptide, a lectin, a lectin fragment, an LDL molecule, an oxidized, modified, chemically treated, heat treated or artificial LDL molecule or portions thereof and/or combinations thereof. Generally, one of the genes may be a GTP-cyclohydrolase I, e.g., a GTP cyclohydrolase I from human, cow, pig, horse, cat, dog, rat, mouse, bear, rabbit, moose, sheep, fish, yeast or fusion proteins thereof.

For uses in vitro, the nucleic acid delivery may be via, e.g., a liposome, an LDL (or derivatives thereof), a nanoparticle, PEG, calcium phosphate precipitation, electroporation, gene injection, a gene gun and combinations thereof. The gene may be under the control of a promoter, e.g., CMV IE, LTR, SV40 IE, HSV tk, β-actin, human globin α, human globin β and/or human globin γ promoter. Another method involves treating a damaged blood vessel in an individual with cardiovascular disease by identifying an individual in need of repair for damaged/dysfunctional endothelial cells and providing to that individual a therapeutically effective amount of a Lox-1 binding agent and a nucleic acid encoding a GTP-cyclohydrolase I associated with a carrier, wherein the Lox-1 binding agent targets the delivery system to Lox-1 expressing cells for delivery of the nucleic acid.

### BRIEF DESCRIPTION OF THE FIGURES

For a more complete understanding of the features and advantages of the present invention, reference is now made to the detailed description of the invention along with the accompanying figures and in which:

FIG. 1 is a graph that shows the acetylcholine-induced relaxation of BBd aortic rings exposed to medium only (Control) or medium containing an adenoviral vector for expressing GTPCH (AdGTPCH) (labeled GTPCH). Other rings were transduced in the same manner but were pretreated with N^{G}-monomethyl-L-arginine (L-NMMA) for 30 minutes prior to addition of acetylcholine (Control-NMMA and GTPCH-NMMA). Data are mean±SEM (with the number of vessel rings indicated in parentheses). The “*” indicates a statistically significant difference (p<0.005) between the response in the GTPCH-infected rings and the control rings at that dose of acetylcholine);

FIG. 2 is a graph that shows the acetylcholine-induced relaxation of BBd aortic rings exposed to medium only (Control—same group as FIG. 1) or medium containing an adenoviral vector for expressing Green Fluorescent Protein (AdGFP) (labeled GFP) as a transduction control. Data are mean±SEM (with the number of vessel rings indicated in parentheses);

FIG. 3 is a graph that shows the acetylcholine-induced relaxation of type II Zucker diabetic fatty (ZDF) rat aortic rings exposed to medium only (Control) or medium containing AdGTPCH vector (GTPCH). Data are mean±SEM (with the number of vessel rings indicated in parentheses). The “*” indicates a statistically significant difference (p<0.05) between the response in the GTPCH-infected rings and the control rings at that dose of acetylcholine;

FIG. 4A through 4C show a correlation of GTPCH expression and acetylcholine-induced vascular relaxation, briefly:

FIG. 4A is a western blot analysis of hemagglutinin-tagged GTPCH protein in cultured cells (positive control) and aortic rings from two different BBd rats (#48 and #60) following the various treatments;

FIG. 4B is a graph that shows the acetylcholine-induced relaxation of AdGTPCH-infected and sham-treated (control) aortic rings from BBd rat #48. The high level of GTPCH expression in BBd rat #48 correlates with increased vessel reactivity (i.e., improved endothelial cell function);

FIG. 4C is a graph that shows the acetylcholine-induced relaxation of AdGTPCH-infected and sham-treated (control) aortic rings from BBd rat #60. The low level of GTPCH expression in BBd rat #60 correlates with lower vessel reactivity (i.e., less improved endothelial cell function); and

FIG. 5 is a diagram that summarizes the biochemical pathway(s) affected by the present invention, briefly, the expression/activity of GTP cyclohydrolase via gene transfer causes a “downstream” increase in tetrahydrobiopterin formation bringing about increased nitric oxide synthesis (an indication of improved endothelial cell function).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

The present inventor has recognized that vascular disease in diabetes is thought to be the result of decreased production of nitric oxide by endothelial cells. Previous work by the inventor showed that nitric oxide production is reduced in diabetes because of a deficiency in tetrahydrobiopterin, an essential cofactor for the enzyme nitric oxide synthase. The tetrahydrobiopterin deficiency, in turn, is the result of decreased expression of GTP-cyclohydrolase I (GTPCH), the rate-controlling enzyme for tetrahydrobiopterin synthesis. Using conventional adenoviral vectors, GTPCH levels were raised in endothelial cells and blood vessels isolated from diabetic animals, increasing tetrahydrobiopterin concentrations, enhancing nitric oxide production, and normalizing vasoreactivity—all signs of improved endothelial function and reversal of diabetic vascular impairment. This demonstration supports using gene therapy to alleviate diabetic vascular disease. However, while viral vectors are effective in gene therapy, the immune response elicited by viral proteins poses a major problem for human use. Additionally, viral vectors are relatively non-selective regarding contact with target cells.

Viral vectors fail to provide the required expression levels, specificity of localization and have caused some safety concerns. Therefore, alternatives to viral gene delivery have been investigated. One such method was discussed by Hood, et al. (Science 296: 2404-2407, 2002), which demonstrated that targeting nanoparticles to the α(v)β(3) integrin expressed on endothelial cells of tumors provided one way to deliver a mutant Raf-1 gene to cause apoptosis of tumor-associated endothelial cells and tumor regression. Hood demonstrated that the targeting ligand could select a subpopulation of endothelial cells, deliver a specific gene to those endothelial cells, affect their biochemical signaling pathways, and have a therapeutic effect. This work is incorporated in U.S. Patent Application No. 20030092655 (published May 15, 2003) which describes a liposome-mediated delivery of genes to angiogenic blood vessels, relevant technical portions incorporated herein by reference. The systems include a cationic amphiphile, a neutral lipid, a targeting lipid, and a nucleic acid complexed with the cationic lipid. However, unlike the construct disclosed therein, the present invention seeks to save or rehabilitate the cell, rather than cause its destruction.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “specific binding” is used to describe the binding that occurs between a paired species such as enzyme/substrate, receptor/agonist, antibody/antigen, and lectin/carbohydrate, which may be mediated by covalent or non-covalent interactions or a combination of covalent and non-covalent interactions. When the interaction of the two species produces a non-covalently bound complex, the binding that occurs is typically electrostatic, hydrogen-bonding or the result of lipophilic interactions. Accordingly, “specific binding” occurs between a paired species that produces a bound complex having the binding and/or specificity characteristics of an antibody/antigen or enzyme/substrate interaction. For example, the specific binding is characterized by the binding of one member of a pair to a particular species and to no other species within the family of compounds to which the corresponding member of the binding member belongs. Thus, for example, an antibody binds preferably to a unique epitope.

The term “targeted” as used herein encompasses the use of antigen-antibody binding, ligand-receptor binding, and other chemical binding interactions to selectively deliver to a target cell, e.g., an endothelial cell, one or more nucleic acids, peptides, polypeptides, drugs, drug precursors and the like that may increase the level of expression, activity, half-life and the like of a gene, e.g., a DNA sequence encoding GTP-cyclohydrolase I. The actual target may be, e.g., Lox-1, a Lox-1-associated protein, a complex that includes Lox-1 or other surface proteins, carbohydrates, lipids and the like that are expressed on endothelial cells. Most often, the target will be expressed primarily on endothelial cells, e.g., dysfunction or damaged endothelial cells.

The term “targeting ligand” is used interchangeably to describe the specific proteins, glycoproteins, carbohydrates, lipids or other molecules that are used in conjunction with the present invention to target a cell surface, e.g., an endothelial cell surface, a dysfunctional endothelial cell or even a cell that naturally or artificially is expressing a cell surface marker to which the targeting ligand binds.

The opposite of the targeting ligand is the “ligand target,” which is the target that is bound specifically by the targeting ligand. By way of example, a “targeting ligand” for use with the present invention is a monoclonal antibody specific for Lox-1 and the “ligand target” is a Lox-1 protein expressed on the cell surface of a target endothelial cell. In another embodiment of the present invention, the targeting ligand is an LDL molecule and its ligand target is an LDL receptor.

As used herein, “nanoparticle” is defined as a particle having a diameter of from 1 to 1000 nanometers, having any size, shape or morphology. The nanoparticle may even be a “nanoshell,” which is a nanoparticle having a discrete dielectric or semiconducting core section surrounded by one or more conducting shell layers. A “nanoshell” is a subspecies of nanoparticles characterized by the discrete core/shell structure. Both nanoshells and nanoparticles may contain dopants for binding to, e.g., negatively charged molecules such as DNA, RNA and the like. Examples of commonly used, positively charged dopands include Pr⁺³, Er⁺³, and Nd⁺³. As used herein, “shell” means one or more shells that will generally surround at least a portion of one core. Several cores may be incorporated into a larger nanoshell. In one embodiment, the nanoparticles are administered to the animal using standard methods. Animals to be treated using the compositions and methods of the invention include, but are not limited to, humans, cows, horses, pigs, dogs, cats, sheep, goats, rabbits, rats, mice, birds, chickens or fish.

As used herein, the term “delivering” nanoparticles is used to describe the placement of the nanoparticles attached to, next to, or sufficiently close to the target location, e.g., intravenously, in order to maximize the number of particles that will be able to contact cells at the target location.

The term “antibody” as used herein, refers to an immunoglobulin molecule, which is able to specifically bind to a specific epitope on an antigen. An antibody may be an IgG, IgM, IgA, IgD and IgE, variants and subclasses thereof. Antibodies may be intact immunoglobulins derived from natural sources or recombinant sources. The antibodies in the present invention may exist in a variety of forms including, for example, polyclonal antibodies, monoclonal antibodies, or even as antibody fragments, e.g., Fab′, Fab, F(ab′)₂, single domain antibodies (DABs), Fv, scFv (single chain Fv), and the like, as well as single chain antibodies and humanized antibodies (Harlow et al., 1988; Houston et al., 1988; Bird et al., 1988). Techniques for preparing and using various antibody-based constructs and fragments are well known in the art. Preparing and characterizing antibodies are also well known in the art (See, e.g., Antibodies: A Laboratory Manual, Cold Spring Harbor Laboratory, 1988; incorporated herein by reference). In one embodiment, the targeting ligand is a monoclonal antibody (MAb), which has certain advantages, e.g., reproducibility and large-scale production. The antibodies may be of: human, murine, monkey, rat, hamster, rabbit or chicken origin. Humanized antibodies are also useful, e.g., chimeric antibodies from mouse, rat, or other species, bearing human constant and/or variable region domains, bispecific antibodies, recombinant and engineered antibodies and fragments thereof. Methods for the development of antibodies that are “custom-tailored” to the target cells are likewise known and such custom-tailored antibodies are also contemplated.

Antibodies may be purified, if desired, using filtration, centrifugation and various chromatographic methods such as HPLC or affinity chromatography. Fragments of the antibodies of the invention may be obtained from the antibodies so produced by methods which include digestion with enzymes, such as pepsin or papain, and/or by cleavage of disulfide bonds by chemical reduction. Alternatively, antibody fragments encompassed by the present invention can be synthesized using an automated peptide synthesizer or by expression of full-length gene or gene fragments in E. coli.

The compositions of the present invention include nucleic acid sequences bound in, to or about nanoparticles, and methods for their use. The bound nanoparticles can be used for the delivery of the nucleic acid sequences to a variety of biological targets, such as endothelial cells. In one embodiment, the nucleic acids, e.g., a nucleic acid gene under the control of a promoter for gene expression is attached to a positively doped nanocore, which is then surrounded by a shell that includes a targeting ligand that is specific for a ligand target on, e.g., an endothelial cell.

One embodiment of the invention relates to nucleic acid sequences bound in/to a nanoparticle. The nanoparticle is prepared by assembly of a “nanoparticle precursor.” The nucleic acid sequence can generally be any nucleic acid sequence selected for delivery into a biological target. The nucleic acid sequence can be DNA, RNA, PNA, or other synthetic or modified nucleic acid sequences. In one embodiment, the nucleic acid sequence is a DNA sequence encoding GTP-cyclohydrolase I (GTPCH). Multiple GTPCH nucleic acid sequences are available, such as rat (GenBank Accession No. NM 024356) and human (GenBank Accession No. NM 000161), relevant portions incorporated herein by reference. The DNA sequence may be a naturally occurring sequence, a modified version of a naturally occurring sequence, or a synthetic sequence. In one embodiment, the nucleic acid is modified to maximize the percentage of codon usage of the target host. The naturally occurring sequence may be a human, cow, pig, horse, cat, dog, rat, mouse, bear, rabbit, moose, fish, sheep, or other animal sequence. The sequence may be modified to add or delete particular sequences. For example, a DNA sequence could be modified to, e.g., remove restriction sites, eliminate common cleavage or mutation sites, maximize binding to a nanocore. The sequence may be further modified to include additional sequences that aid in transcription, translation, localization, elimination of protein cleavage sites, addition of cleavage and/or processing sites, and addition or removal of glycosylation sites.

In one embodiment, the nanoparticle precursor includes a nucleic acid sequence bound to a nanoparticle polymer. The bond between a nanoparticle precursor and a nucleic acid may be non-covalent or covalent. The nanoparticle polymer may be any polymer that can assemble into a nanoparticle. For example, the nucleic acid sequence can be non-covalently bound to a first polymer. This first polymer can be a DNA binding cationic polymer such as polyethyleneimine (“PEI”). The first polymer can be covalently bound to a second polymer. The second polymer can be a hydrophilic polymer such as polyethylene glycol (PEG). For example, the second polymer can be conjugated to a fraction of the primary amines of PEI.

The hydrophilic polymer can be bound to a ligand such as an antibody. The antibody can be a polyclonal antibody or a monoclonal antibody, with a monoclonal antibody being presently preferred. The antibody can be specific for a biological receptor or other cellularly expressed protein. For example, the antibody can bind the lectin-like oxidized low density lipoprotein (LDL) receptor-1, Lox-1. Antibodies provide attractive binding abilities, but have relatively high steric bulk. Smaller antibody fragments or other binding peptides or molecules may be used as a ligand in various embodiments of the invention.

When the nanoparticle precursor self-assembles, the nucleic acid molecule is encapsulated within the formed nanoparticle, and the antibody or ligand is presented on the surface of the nanoparticle. The encapsulated nucleic acid molecule is partially or fully protected from degradation by the environment, enzymes, hydrolysis, or other degrading forces.

The assembled nanoparticle can generally have an average diameter of about 1 nm to about 1000 nm. More narrow ranges of diameters include about 10 nm to about 250 nm, and about 40 nm to about 100 nm. In one embodiment, the nucleic acid sequence is a DNA sequence encoding GTP-cyclohydrolase I; the nucleic acid sequence is non-covalently bound to PEI (first polymer), PEI is covalently bound to PEG (second polymer), and PEG is covalently bound to a monoclonal antibody that binds the lectin-like oxidized low density lipoprotein (LDL) receptor-1 (Lox-1) at the end opposite from the first polymer.

An additional embodiment of the invention relates to the assembled nanoparticle. The assembled nanoparticle comprises nanoparticle precursors that have assembled in solution. The assembled nanoparticles preferably contain nucleic acid sequences in the internal volume of the nanoparticles, and antibodies or other binding peptides presented on the external face of the nanoparticles. The nanoparticles can generally be any shape, with about spherical being presently preferred. The antibodies preferably maintain their natural conformation, allowing binding to their natural targets.

The assembled nanoparticles can be present in a variety of formulations including in solution, dried, in liposomes, and so on. Specific examples of formulations include fullerene nanoparticles, aqueous nanoparticles comprised of oppositely charged polymers polyethylenimine (PEI) and dextran sulfate (DS) with zinc as a stabilizer, calcium phosphate nanoparticles, end-capped oligomers derived from Tris(hydroxymethyl)aminomethane bearing either a hydro- or a fluorocarbon tail; conjugated poly(aminopoly(ethylene glycol)cyanoacrylate-co-hexadecyl cyanoacrylate (poly(H(2)NPEGCA-co-HDCA) nanoparticles, biodegradable nanoparticles formulated from poly (D,L-lactide-co-glycolide) (PLGA), and water soluble, biodegradable polyphosphoester, poly(2-aminoethyl propylene phosphate) (PPE-EA) nanoparticles.

Aspects of the invention also relate to methods of preparing the assembled nanoparticles. The methods can comprise formation of a polymer conjugate, and contacting the polymer conjugate with nucleic acid to form a nanoparticle. The polymer conjugate includes a first polymer, a second polymer, and a ligand. The first polymer preferably binds in a non-covalent manner to nucleic acids. A presently preferred first polymer is a DNA binding cationic polymer such as polyethyleneimine (“PEI”). The second polymer can be a hydrophilic polymer such as polyethylene glycol (“PEG”). The ligand is presently preferred to be an antibody.

It is presently preferred that the parts of the polymer conjugate be connected by covalent bonds. The specific order of assembly of the polymer conjugate can be varied. For example, the first polymer and second polymer can be connected, then the ligand can be connected. Alternatively, the second polymer and the ligand can be connected, then the first polymer can be connected. The methods can further comprise an isolation or purification step to be performed after the contacting step.

The above described assembled nanoparticles can be used in a variety of applications. The nanoparticles can be used in in vitro or in vivo applications. One embodiment of the invention relates to use of the assembled nanoparticles to deliver a DNA sequence encoding GTP-cyclohydrolase I (GTPCH) to damaged endothelial cells. This delivery promotes long-term production (“expression”) of the GTPCH protein in endothelial cells. This delivery is expected to be beneficial in treating both type I (insulin-dependent) and the more prevalent type II (insulin-resistant) diabetes. In addition, this delivery is expected to improve endothelial cell dysfunction associated with cardiovascular disease linked to diabetes, hypertension, dyslipidemia and smoking.

Endothelial cells have been shown to express the Lox-1 receptor on their surface in response to the oxidative damage and dyslipidemia associated with diabetes (Chen M., et al. Biochem. Biophys. Res. Commun. 287: 962-968, 2001). Targeting of the assembled nanoparticles to damaged endothelial cells is accomplished by use of a monoclonal antibody that binds the lectin-like oxidized low density lipoprotein (LDL) receptor-1 (Lox-1). Increased levels of GTPCH protein in endothelial cells has been shown to increase tetrahydrobiopterin levels, increase nitric oxide synthesis and reverse endothelial dysfunction caused by disease (Meininger, C. J., et al. FASEB J., in press, 2004), in addition to increasing the antioxidant capabilities of endothelial cells to prevent future damage/dysfunction (Alp, N. J., et al., J. Clin. Invest. 112:725-735, 2003).

The assembled nanoparticles preferably deliver the nucleic acid sequence selectively to damaged endothelial cells. Preliminary work to demonstrate the selectivity of nucleic acid transfer will involve the use of a nanoparticle containing a nucleic acid encoding a protein that can be assayed in cells and tissues, such as luciferase or beta galactosidase, or one that can be visualized, such as green fluorescent protein. The assembled nanoparticles can be administered in in vivo applications via intravenous injections. The administration can include a single administration, periodic administration, or continuous administration.

The administration preferably reverses or ameliorates damage to endothelial cells caused by diabetes or other disease conditions such as smoking, dyslipidemia, hypertension, and cardiovascular disease. The amelioration of damage preferably is at least about 30%, about 40%, about 50%, about 60%, about 70%, about 80%, about 90%, or ideally about 100%. Repair of damage can be assessed by measuring the ability of the endothelial cells to synthesize nitric oxide and to perform their normal biological functions, e.g., vasodilatation, proliferation, and so on.

Diabetes mellitus is characterized by a loss of nitric oxide (NO) bioactivity, resulting in endothelial dysfunction (1). Since NO plays an important role in maintaining vascular homeostasis, the loss of NO bioactivity is thought to be an early step in the development of vascular complications associated with this disease (1). Synthesis of NO in endothelial cells is impaired in diabetes (2). As a result, endothelium-dependent relaxation is also impaired (3) and likely contributes to development of cardiovascular disease in diabetic patients (4). However, the factors contributing to NO-mediated endothelial dysfunction in diabetes are not fully defined.

Impaired NO production in coronary endothelial cells of the spontaneously diabetic BB (BBd) rat (a model of human type I diabetes) is due to a deficiency of tetrahydrobiopterin (BH4), a cofactor necessary for NO synthesis by endothelial NO synthase (eNOS) (5). The BH4 deficiency is the result of decreased expression/activity of GTP cyclohydrolase I (GTPCH), the first and rate-controlling enzyme in de novo biosynthesis of BH4. Without adequate BH4, NO synthesis by eNOS is impaired. BH4 levels in endothelial cells can be modulated by targeting the BH4 biosynthetic pathway using pharmacological agents (6). Increasing BH4 concentrations in endothelial cells from diabetic rats increased NO synthesis. Using a recombinant adenovirus encoding human GTPCH (AdGTPCH), viral gene delivery can be used to increase BH4 and enhance NO production in a dose-dependent, eNOS-specific manner in cultured endothelial cells and isolated vessels from models of human type I or type II diabetes exhibiting endothelial dysfunction and impaired NO synthesis. Vascular reactivity is significantly improved by GTPCH gene transfer.

One type of nanoparticle for use with the present invention is taught by, e.g., U.S. Pat. No. 6,530,944, issued to West, et al., which teaches basic methods for the manufacture, manipulation and use of nanoparticles and nanoshells, relevant portions incorporated herein by reference. For example, a metal nanoshell can be made using well-known principles of molecular self-assembly and colloid chemistry in aqueous solution. Conceptually, the method is straightforward and includes: growing or obtaining silica nanoparticles dispersed in solution, for example, the silicon dioxide particles such as LUDOX TM-50 colloidal silica particles (Aldrich Chemical Co., Milwaukee, Wis.); attaching very small (1-2 nm) metal “seed” colloids to the surface of the nanoparticles via molecular linkages (these seed colloids cover the dielectric nanoparticle surfaces with a discontinuous metal colloid layer); and growing additional metals onto the “seed” metal colloid adsorbates via chemical reduction in solution. The nanoparticles may be made positively charged by doping the core and/or the shell with, e.g., rare earth ions such as, e.g., Neodymium, Erbium and/or Praseodymium. These same metals may be used as attachment points for chemical links and/or tethers for the ligand targeting molecule, e.g., an antibody or peptide or lipoprotein.

The nanoshell taught by West may be used in conjunction with the present invention to deliver an amount effective to deliver a nucleic acid that overcomes a downstream NO deficiency. The nanoshell and the targeting ligand are used to deliver, the one or more genes to cell(s), tissue(s) or organism(s) with the nanoshell to produce a desired therapeutic benefit. The therapeutic effect may be achieved by delivering to the cell, tissue or organism with a single composition or pharmacological formulation that includes the nanoshell and one or more genes, or by contacting an endothelial cell with two or more distinct compositions or formulations, wherein one composition includes a nanoshell and the other includes one or more agents.

Examples of linker molecules include: aminopropyltriethoxysilane, aminopropyltrimethoxysilane, diaminopropyldiethoxysilane, or 4-aminobutyl dimethylmethoxysilane and the like. In addition, the surface may be terminated with a linker that allows for the direct reduction of metal atoms on the surface rather than through a metallic cluster intermediary. In other embodiments, reaction of tetrahydrothiophene (AuCl) with a silica core coated with diphenyltriethoxysilane leaves a surface terminated with gold chloride ions that may provide sites for additional gold reduction. Alternatively, a thin shell of another nonmetallic material, such as CdS or CdSe grown on the exterior of a silica particle permits a metallic shell to be reduced directly onto the nanoparticle's surface. In other embodiments, functionalized oligomers of conducting polymers may be attached in solution to the functionalized or non-functionalized surface of the core nanoparticle and subsequently cross-linked by thermal or photo-induced chemical methods. The nanoparticle or shell may then be further coated with one or more polymers, liposomes, LDL or LDL derivatives and the like.

United States Patent Application No. 20030013674, by Bednarski, et al., teaches basic techniques for the use of targeted cross-linked nanoparticles for in vivo gene delivery, relevant portions incorporated herein by reference. Briefly, this application teaches basic in vivo delivery of nucleic acids enhanced by delivery of the nucleic acid in a complex with nanoparticles; where the nanoparticles comprise cross-linked neutral amphipathic molecules, cationic amphipathic molecules and targeting amphipathic molecules. Optionally, the application teaches use of cationic and targeting amphipathic molecules that are cross-linked. The targeting moiety present on the targeting amphipathic molecule provides for selective delivery of the complex to a predetermined target site, e.g., blood vessels, tumor cells, liver cells, and the like.

Bednarski teaches the amount of DNA/nanoparticle complex required to accomplish expression of a desired gene product at an effective level. Generally, the amount of DNA/nanoparticle complex administered is an amount sufficient to provide for transformation of a number of cells that in turn provides for a level of gene product expression from the introduced DNA/nanoparticle complex to provide for a desired effect. In the case of Bednarski, et al., the effect is destruction of the targeted cell. In the case of the present invention, the DNA/nanoparticle is used to target Lox-1 expressing endothelial cells such that a deficiency in tetrahydrobiopterin, an essential cofactor for the enzyme nitric oxide synthase, is reduced or eliminated and the cell is rescued/protected from disease-induced damage, dysfunction, and/or death. Tetrahydrobiopterin deficiency results from, e.g., a decreased expression of GTP-cyclohydrolase I (GTPCH), the rate-controlling enzyme for tetrahydrobiopterin synthesis. The present invention combines a novel targeting ligand, the expression of a gene that overcomes the tetrahydrobiopterin deficiency and the use of nanoparticles. Dosages are routinely determined in the art, and can be extrapolated from the amounts of DNA/nanoparticle complex effective in an animal model (e.g., a rodent (mouse or rat) or other mammalian animal model), in which factors such as the efficiency of transformation and the levels of gene product expression achieved may be readily assessed and extrapolated to other vertebrate subjects.

The basic steps for DNA/nanoparticle formation and delivery are taught by Bednarski as: forming cross-linked nanoparticles (NPs) by self-assembly and polymerization of the appropriate amphipathic molecules. Instead of using a complicated trivalent lipid-integrin ligand, the present invention uses anti-Lox-1 antibodies or other Lox-1-binding agents to target and deliver the nucleic acids to rescue the cells, rather than killing them by inducing apoptosis. For example, a diacetylene phospholipid is mixed in a chloroform solution and an anionic chelator lipid is added. The surface density of the anti-Lox-1 antibody on the NPs may be controlled by varying the concentration of antibody. To form nanoparticles, the combined lipid solution is evaporated to dryness and dried under high vacuum to remove any residual solvent. The dried lipid film is hydrated using deionized water and the suspension is sonicated at temperatures above the gel-liquid crystal phase transition, e.g., 65° C., for 1 hour, while maintaining the pH between 7.0 and 7.5 using, e.g., a low concentration sodium hydroxide solution. The vesicles may then be cross-linked by cooling the solution on ice and irradiating the solution at 254 nm with a UV lamp, as required. The DNA is then added to the cross-linked nanoparticles and the efficiency of delivery measured using a reporter gene, e.g., a green fluorescent protein (GFP) after allowing the particles to interact with target cells, e.g., Lox-1 expressing endothelial cells.

Yet another technique for use with the present invention is taught in United States Patent Application 20030092655, issued to Cheresh, et al., in which integrin receptor targeting liposomes that include a cationic amphiphile such as a cationic lipid, a neutral lipid, and a targeting lipid are used to deliver nucleic acids, relevant portions incorporated herein by reference. Unlike Cheresh, the present invention delivers one or more genes that overcome a deficiency in intracellular tetrahydrobiopterin concentration caused by disease, ameliorating disease-induced dysfunction and providing cells with protection from future disease-induced damage, dysfunction and/or death.

The following examples are included to demonstrate supporting data and preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example I

Nitric Oxide (NO) Synthesis in Endothelial Cells is Impaired in Diabetes.

As shown herein, the inhibition of NO synthesis in the spontaneously diabetic BB (BBd) rat is due to decreased levels of tetrahydrobiopterin (BH4), secondary to decreased expression of GTP cyclohydrolase I (GTPCH). In one example, an adenoviral GTPCH gene transfer was used to reverse BH4 deficiency and repair the ability of endothelial cells to produce NO. GTPCH gene transfer increased BH4 levels in BBd endothelial cells from 0.17±0.02 (mean±SEM) to 73.37±14.42 pmoles/million cells and NO production from 0.77±0.07 to 18.74±5.52 nmole/24 hr/million cells.

To demonstrate a functional effect of increasing BH4 concentrations in tissues, GTPCH was transferred into aortic rings from BBd and Zucker diabetic fatty (ZDF) rats, models of human type I and type II diabetes, respectively. GTPCH gene transfer led to a dose-dependent increase in acetylcholine-induced vasorelaxation, preventable by inhibiting NO synthase. Maximal relaxation of virus-treated rings (10¹⁰ virus particles/ml) to acetylcholine was significantly higher than sham-treated rings (BBd 64% vs. 37%, p<0.005; ZDF 80% vs. 44%, p<0.05). This study demonstrates GTPCH gene transfer can reverse BH4 deficiency in both type I and type II diabetes and provides a basis for using gene therapy to treat cardiovascular complications in diabetic patients.

METHODS. Animals. Male diabetic BB (BBd) rats were obtained from the Animal Resources Division of the Health Protection Branch (Ottawa, Canada). Male Zucker diabetic fatty (ZDF) rats were obtained from Charles River (Wilmington, Mass.).

Adenovirus Vectors. A hemagglutinin-tagged human GTPCH cDNA was cloned into the pShuttleCMV plasmid and used to generate a recombinant adenovirus, AdGTPCH, as previously described (7). A control recombinant adenovirus, AdGFP, encoding green fluorescent protein (but containing no hemagglutinin tag), was generated using the same system.

Transduction of Cultured Cells: Coronary endothelial cells were isolated from BBd rats as previously described (5,6). Aortic smooth muscle cells (Cell Applications, San Diego, Calif.) were plated at 70-80% confluence in 100 mm dishes and cultured overnight. Cells were exposed to an adenoviral vector (moi=50) in 1 ml of growth medium containing 2% fetal bovine serum and 0.4 mM L-glutamine for 1 hr. This virus-containing medium was then diluted by the addition of 3 mls of growth medium containing 10% serum/0.2 mM L-glutamine for 3 hrs. Finally, another 3 mls of the same medium was added to the dishes for 20 hrs. Medium was then replaced and cells were incubated an additional 24 hrs before being extracted for BH4 analysis. In some studies, 2,4-diamino-6-hydroxypyrimidine (DAHP, 10 mM), a GTPCH inhibitor, was added to the culture medium 30 minutes before the addition of AdGTPCH and was maintained throughout the culture period.

BH4 Assay: To measure levels of BH4, the HPLC method of Fukushima and Nixon (8) was modified, as previously described (Meininger C. J., Marinos R. S., Hatakeyama K, Martinez-Zaguilan R., Rojas J. D., Kelly K. A., and Wu G. (2000) Impaired nitric oxide production in coronary endothelial cells of the spontaneously diabetic BB rat is due to tetrahydrobiopterin deficiency. Biochem. J. 349, 353-356, relevant portions incorporated herein by reference). Briefly, endothelial cells (3-5×10⁶ cells) were washed and suspended in 0.3 ml of 0.1 M phosphoric acid containing 5 mM dithioerythritol (an antioxidant), to which 35 μl of 2 M trichloroacetic acid (TCA) was added. The solution was centrifuged and the supernatant was used immediately for BH₄ analysis. For oxidation under acidic conditions, 100 μl of cell extract or BH₄ standard (50 pmol/ml) was mixed with 15 μl of 0.2 M TCA and 15 μl of 1% I₂/2% KI in 0.2 M TCA. For oxidation under alkaline conditions, 100 μl of cell extract or BH4 standard (50 pmol/ml) was mixed with 15 μl of 1 M NaOH and 15 μl of 1% I/2% KI in 3 M NaOH. The amount of BH4 in the endothelial cell extracts was calculated from the difference between the amount of biopterin formed by oxidation at acidic conditions and the amount of biopterin formed by oxidation at alkaline conditions.

NO Synthesis by Endothelial Cells. For assessing NO synthesis, conditioned media were analyzed for nitrite plus nitrate (stable metabolites of NO) using a sensitive fluorometric HPLC method developed by the present inventor (Li H., Meininger C. J., and Wu G. (2000) Rapid determination of nitrite by reversed-phase high performance liquid chromatography with fluorescence detection. J. Chromatogr. B 746, 199-207, relevant portions incorporated herein by reference). Briefly, conditioned medium was incubated with 316 μM 2,3-diamnonaphthalene, followed by addition of 2.8 M NaOH. The 2,3-naphthotriazole formed from the reaction with nitrite was separated on a reversed-phase C₈ column. Nitrate in culture medium was measured by this method after its conversion to nitrite using nitrate reductase. Culture medium without cells was used as a blank.

Ex Vivo Gene Transfer in Vessel Segments: Thoracic aortic rings (3-4 mm in length) from BBd and ZDF rats were transduced with adenovirus vectors (10¹⁰ virus particles/ml) in Dulbecco's modified Eagle's medium containing 0.5% bovine serum albumin for 4 hrs, rinsed, incubated for an additional 20 hrs in the same medium, then used for vessel reactivity studies.

Vessel Reactivity Studies: Endothelium-dependent vasodilation was assessed as previously described (Griffin K. L., Laughlin M. H., and Parker J. L. (1997) Exercise training improves endothelium-mediated vasorelaxation after chronic coronary occlusion. J. Appl. Physiol. 87, 1948-1956, relevant portions incorporated herein by reference). Briefly, thoracic aortic rings were carefully mounted on two stainless steel wires, one attached to a force transducer and the other attached to a micrometer. After mounting, the rings were lowered into a bath containing Krebs bicarbonate buffer, equilibrated for 30 minutes, and systematically stretched to the optimum of the length-active tension relationship. Rings were then pre-constricted with norepinephrine and the concentration-response relationships to acetylcholine (10⁻¹⁰-10⁻⁴M) were determined by cumulative addition of acetylcholine in half-log increments directly to the bath.

Immunoblot Procedure. Cellular protein from transduced BBd endothelial cells and BBd vascular rings was analyzed by western blot for hemagglutinin expression, as previously described (7).

Statistical Analysis: Cultured cell data were analyzed by one-way ANOVA with Student-Newman-Keuls test for identifying differences among means. Concentration-response curves were compared using two-way ANOVA for repeated measures. Differences between individual points were ascertained using Fisher's test for least significant difference. Statistical significance was defined as p<0.05.

The present invention was developed to reverse or retard endothelial dysfunction in order to modify the natural history of diabetic vascular disease. An adenoviral vector AdGTPCH was used to transfer the gene for GTPCH in order to modulate intracellular BH4 levels in cultured BBd coronary endothelial cells and in isolated vascular rings from both BBd and ZDF rats. Enhancing de novo BH4 biosynthesis increased NO production and improved vessel reactivity, signs of improved endothelial function.

Cultured BBd endothelial cells expressed only 0.17±0.02 pmoles/million cells of BH4 (Table 1). Following AdGTPCH infection, BH4 concentrations increased over 400-fold (73.37±14.42). This increased BH4 biosynthesis was the result of GTPCH gene transfer, as transduction with control virus (AdGFP) did not significantly increase BH4 levels (0.26±0.04, p>0.05). Furthermore, blocking GTPCH activity with 10 mM DAHP prevented the increase in BH4 level brought about by the GTPCH gene transfer (0.18 pmoles/million cells with DAHP vs. 0.17 pmoles/million cells without DAHP). The rise in BH4 concentration brought about by AdGTPCH infection was sufficient to increase NO production by the same cells (18.74±5.52 nmole/24 hr/million cells compared to 0.77±0.07 in control cells) (Table 1, below). The production of NO was not significantly increased in AdGFP-infected endothelial cells (2.77±0.61, p>0.05). No production of NO could be detected if the cells were treated with DAHP before AdGTPCH infection (data not shown).

TABLE 1

| | | NO Production |
|---|---|---|
| | BH4 Concentration | (nmole/24 hr/ |
| Cell Group | (pmole/million cells) | million cells) |
| BBd EC | | |
| Control | 0.17 ± 0.02 | 0.77 ± 0.07 |
| GFP virus-transduced | 0.26 ± 0.04 | 2.77 ± 0.61 |
| GTPCH virus-transduced | 73.37 ± 14.42* | 18.74 ± 5.52* |
| Aortic SMC | | |
| Control | N.D. | 0.13 ± 0.01 |
| GTPCH virus-transduced | 69.70 ± 3.90* | 0.16 ± 0.01 |

BBd EC, BBd endothelial cells; Aortic SMC, rat aortic smooth muscle cells.
N.D., none detected.
Data are means ± SEM, n = 5 for BBd EC and n = 3 for aortic SMC.
*P < 0.01 vs. control and AdGFP-transduced group.
GFP group is not significantly different from control, p > 0.05.

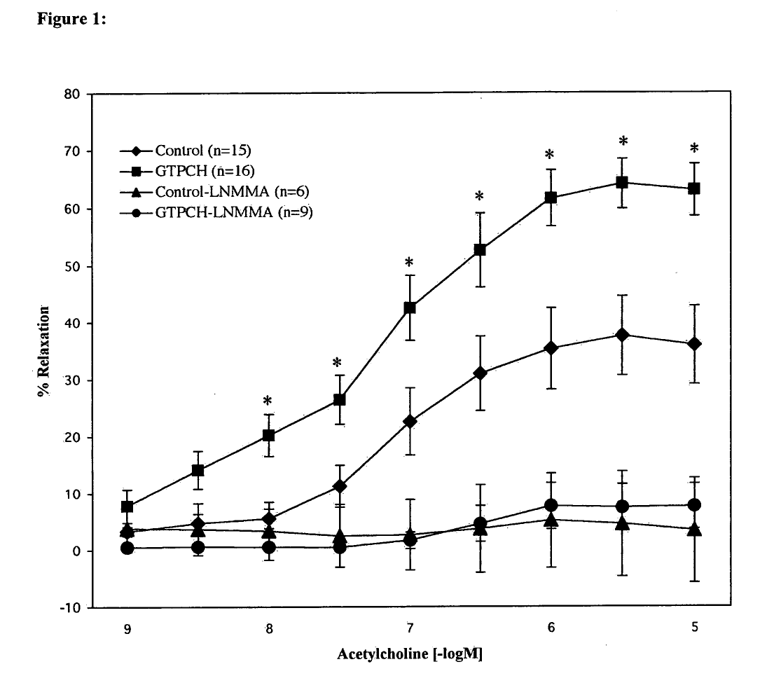

To demonstrate a similar benefit in functional tissues, ex vivo GTPCH gene transfer was used to increase BH4 and thus NO synthesis in isolated vascular rings from BBd rats. BBd vascular rings incubated with AdGTPCH virus showed significantly increased vasodilatory responses to acetylcholine (FIG. 1). The vasodilation was due entirely to NO synthesis as no acetylcholine-induced relaxation occurred when rings from the same vessel were pretreated with N^{G}-monomethyl-L-arginine (L-NMMA; 100 μM), an arginine analogue that blocks NO synthesis.

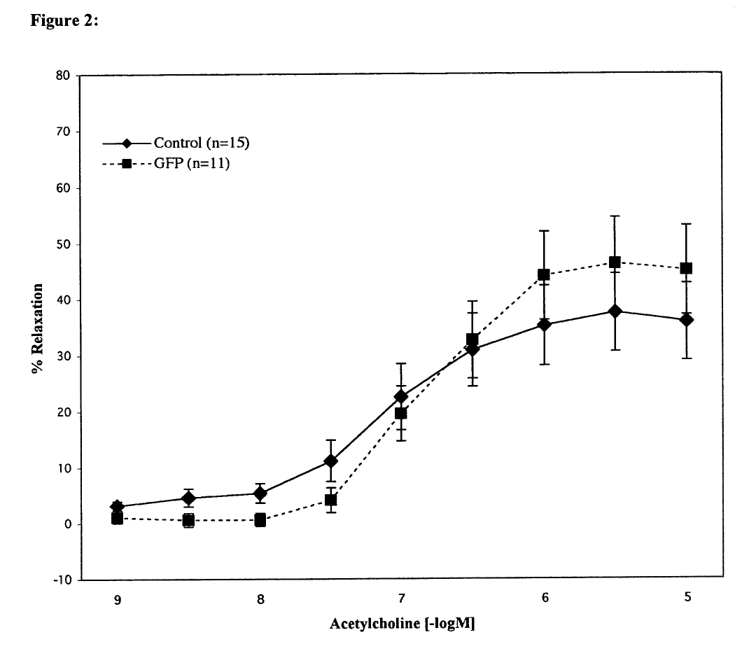

The maximal vasodilatory response of AdGTPCH-treated BBd rings (10¹⁰ virus particles/ml) to acetylcholine (10 μM) was significantly higher than the response of sham-treated (control) rings (64% vs. 37%, p<0.005). Rings infected with AdGFP control virus (GFP) showed no significant increase in relaxation compared to sham-treated control rings (p>0.05, FIG. 2), indicating that transfer of the GTPCH gene was responsible for increased vasorelaxation.

Because virus may enter either endothelial cells or smooth muscle cells in the vessel rings, the effect of GTPCH gene transfer into cultured rat aortic smooth muscle cells was investigated. No BH4 could be detected by our assay in control smooth muscle cells (i.e., no virus exposure) (Table 1). Following AdGTPCH infection, BH4 levels in smooth muscle cells increased to a level equivalent to what was observed in cultured BBd endothelial cells exposed to the virus (69.70±3.90 vs. 73.37±14.42 pmole/million cells). Interestingly, the transfer of GTPCH into smooth muscle cells did not lead to a significant increase in NO synthesis in these cells. NO production was 0.13±0.01 nmole/24 hr/million cells in control cells vs. 0.16±0.01 nmole/24 hr/million cells in AdGTPCH-treated cells. This is in contrast to BBd endothelial cells, which exhibited approximately a 25-fold increase in NO production following GTPCH gene transfer (Table 1).

Endothelial dysfunction occurs in both type I and type II diabetes. To determine if endothelial cells from ZDF rats exhibit a deficiency in BH4 levels, similar to endothelial cells from the BBd rats, we isolated coronary endothelial cells from the ZDF rats before the onset of diabetes at 7 weeks (i.e., cells collected at 6 weeks of age) as well as two weeks and twelve weeks after the onset of diabetes (9 weeks and 19 weeks of age, respectively) and compared them to endothelial cells from age-matched lean control rats. A significant decrease in BH4 levels was evident within two weeks of disease onset and worsened with increasing duration of diabetes (Table 2). Hyperglycemia, also evident after two weeks of diabetes, was maintained for the duration of the study (Table 2).

TABLE 2

| | | | Disease | Plasma | | |
|---|---|---|---|---|---|---|
| | | Age | Duration | Glucose | Body Weight | BH₄ Concentation |
| Rat Group | | (weeks) | (weeks) | (mM) | (g) | (pmoles/10⁶ cells) |
| Lean | (n = 5) | 6 | 0 | 7.56 ± 0.28 | 151.4 ± 2.4 | 0.85 ± 0.03 |
| Obese | (n = 5) | 6 | 0 | 7.92 ± 0.32 | 177.2 ± 2.8** | 0.82 ± 0.02 |
| Lean | (n = 5) | 9 | 2 | 7.42 ± 0.26 | 252.8 ± 33 | 0.82 ± 0.02 |
| Obese | (n = 5) | 9 | 2 | 24.4 ± 0.69** | 318.0 ± 3.8** | 0.64 ± 0.02* |
| Lean | (n = 5) | 19 | 12 | 7.36 ± 0.27 | 389.0 ± 4.1 | 0.80 ± 0.04 |
| Obese | (n = 6) | 19 | 12 | 26.8 ± 0.56** | 407.5 ± 4.3* | 0.42 ± 0.02** |

Cells are freshly isolated and analyzed immediately.
BH4 data are means ± SEM, n = 5.
*p < 0.05 vs. age-matched lean controls,
**p < 0.01 vs. age-matched lean controls, as analyzed by unpaired t-test.

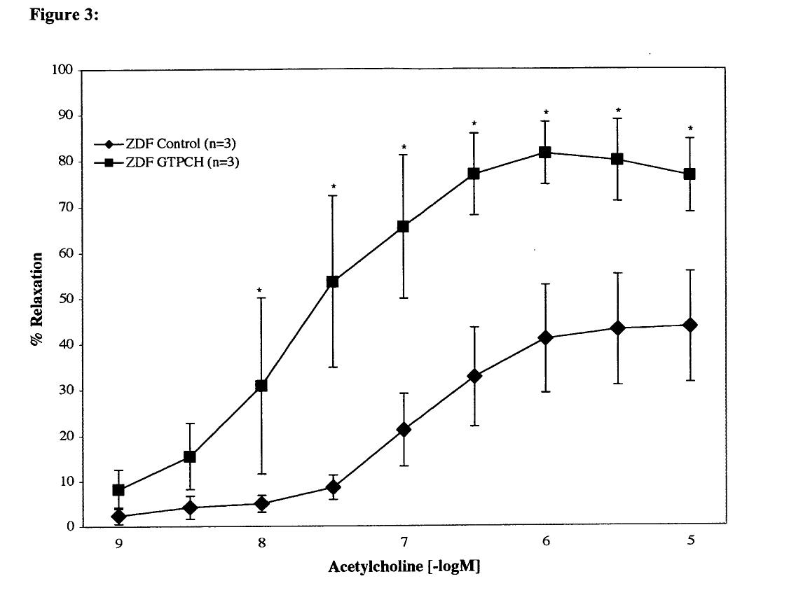

When aortic rings from ZDF rats were infected with the GTPCH-containing virus, vasodilatory responses to acetylcholine were also significantly (p<0.05) increased (FIG. 3). The maximal dilatory response increased from 44% in the sham-treated ZDF rings (control) to 80% in the AdGTPCH-treated rings, again indicating a beneficial effect of increased GTPCH expression. No response to acetylcholine was observed if vessels were pretreated with L-NMMA (data not shown), indicating that the increase in the vasodilatory response to acetylcholine occurs via an increase in NO synthesis.

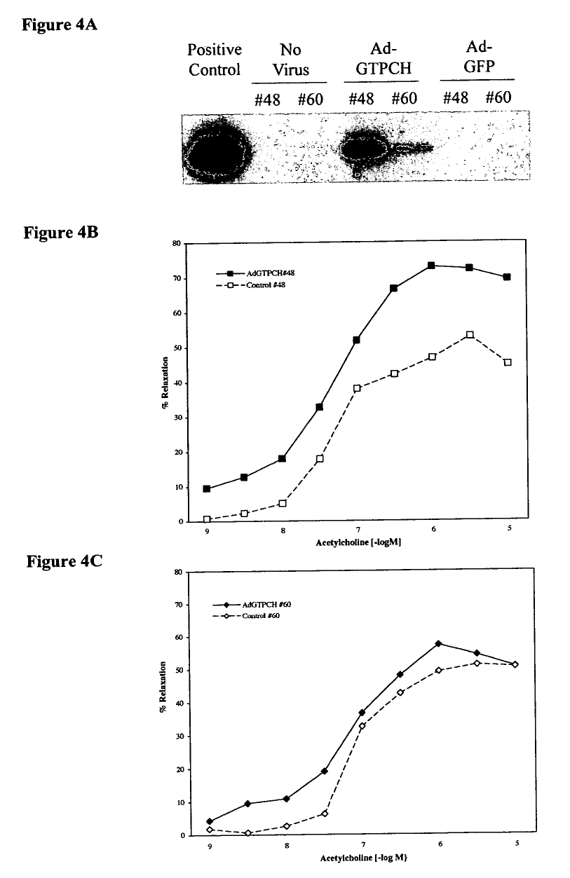

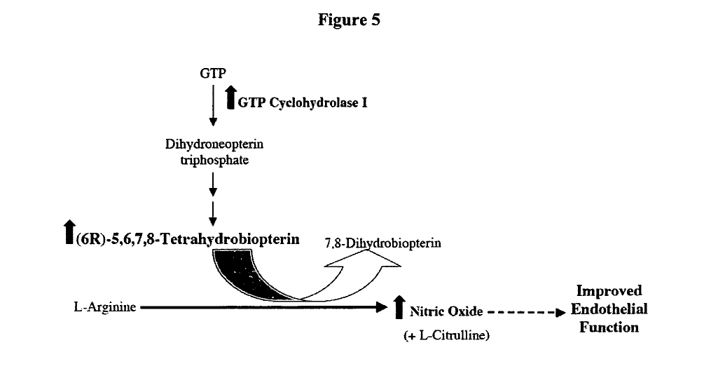

GTPCH protein levels were evaluated in aortic rings, following the acetylcholine dose-response studies, by examining the differential hemagglutinin-tagged GTPCH expression resulting from virus infection. Representative examples of BBd rings are shown in FIG. 4A. Non-virus-treated vessel lysates (lanes 2 and 3) or AdGFP-treated (transduction control) vessel lysates (lanes 6 and 7) exhibited no hemagglutinin, as expected. Hemagglutinin-tagged GTPCH expression was easily demonstrated in AdGTPCH-infected vessels (lanes 4 and 5). Importantly, the degree of relaxation brought about by GTPCH gene transfer directly correlated with the level of GTPCH protein expression in the vessel (FIGS. 4B and 4C [vascular relaxation curves for rings used for protein lysates in FIG. 4A]). A vascular ring with high GTPCH expression (GTPCH #48) relaxed more than a ring with low GTPCH expression (GTPCH #60).

Vascular disease represents a major cause of morbidity and mortality associated with diabetes mellitus. Endothelial dysfunction underlies the vascular complications associated with this disease and represents a therapeutic target for prevention and treatment of vascular disease. The feasibility of modulating BH4 levels via gene transfer in order to reverse endothelial dysfunction accompanying both type I and type II diabetes was investigated.

BH4 is absolutely essential for the formation of NO. Without its presence, NO cannot be synthesized. Supplementation with exogenous BH4 has been shown to improve endothelium-dependent relaxation to acetylcholine in aortic rings from streptozotocin-induced diabetic rats (11), small mesenteric arteries from spontaneously diabetic (db/db −/−) mice (12), and human blood vessels from type II diabetic patients (13), suggesting that endothelial dysfunction in diabetes may be linked to BH4 deficiency.

The present inventor has shown that BH4 levels are deficient in endothelial cells of BBd rats, leading to severely impaired NO synthesis. The consequence of this impaired NO production is decreased endothelial cell proliferation, presumably affecting wound healing, and decreased vascular reactivity. A BH4 deficiency is evident in freshly isolated endothelial cells from both BBd rats (our unpublished observations) and rats made diabetic by treatment with streptozotocin (15), indicating that the deficiency is not an artifact due to culturing of cells. These studies also demonstrate that BH4 levels are decreased in freshly isolated endothelial cells of the ZDF rat, a model of type II diabetes.

Male ZDF rats, a commonly used model of impaired insulin sensitivity, have a defective leptin receptor leading to hyperphagia and obesity. As a result of obesity and subsequent insulin resistance, ZDF rats develop type II diabetes at the age of 7-8 weeks of age and go on to develop severe hyperglycemia comparable to that in the streptozotocin-induced and spontaneously diabetic BBd rat models of type I diabetes. At 7 weeks of age, plasma glucose levels become significantly higher in ZDF rats compared to lean controls and continue to rise in subsequent weeks, while plasma glucose levels remain unchanged in the lean rats (16, our unpublished observations). Obesity in ZDF rats precedes development of hyperglycemia. The appearance of a BH4 deficiency in these animals correlated with the onset of hyperglycemia, not obesity, and worsened with the duration of disease (Table 2).

BH4 is formed in endothelial cells by two pathways: the de novo pathway (which controls synthesis of BH4 from GTP) and a salvage pathway (which involves reduction of intracellular dihydrobiopterin to BH4). In the rat, the first and rate-controlling enzyme in the de novo pathway is GTPCH. Without sufficient GTPCH expression/activity in the endothelial cell, BH4 levels drop and NO synthesis is impaired. One of the biological consequences of this decrease in NO synthesis is impaired vessel reactivity (14). Mitchell, et al., demonstrated that pharmacological inhibition of GTPCH by DAHP impairs endothelium-dependent relaxation and increases blood pressure (17). Incubation of these vessels with sepiapterin, which raises BH4 via the salvage pathway, restored vascular reactivity to acetylcholine. These data support the dependence of the endothelial cell on GTPCH activity to supply BH4 for NO synthesis and normal vessel reactivity. Indeed, when BBd vessels transduced with AdGTPCH were pretreated with 10 mM DAHP, BH4 levels did not rise and an increase in NO production was not detected. Thus, GTPCH gene transfer augments BH4 concentration, allowing increased NO synthesis in diabetic-rat endothelial cells.

Aortic rings from BBd rats were previously shown to exhibit decreased vasodilation in response to acetylcholine (an NO-dependent vasodilator) while maintaining the vascular smooth muscle vasodilatory response to NO donors, indicating endothelial but not smooth muscle dysfunction (11, 14). GTPCH gene transfer, in blood vessels from animals shown to exhibit a deficiency in BH4 (both BBd and ZDF rats), causes a significant increase in the endothelium-dependent vasodilatory response to acetylcholine. This vascular relaxation, however, only occurred in the absence of L-NMMA (FIG. 1), indicating that GTPCH was supporting NO production, presumably via an increase in BH4 concentrations. GTPCH gene transfer in cultured endothelial cells from the diabetic BB rat increased BH4 levels leading to an increase in NO production, similar to what was demonstrated in nondiabetic human endothelial cells (7). This increase in NO synthesis is the basis for the GTPCH-induced reversal of impaired vessel reactivity in the diabetic animals.

Zheng et al. reported similar results following GTPCH gene transfer in a non-diabetes disease model (18). GTPCH gene transfer in the hypertensive deoxycorticosterone acetate (DOCA)-salt rat restored arterial GTPCH activity and BH4 levels in carotid arteries, resulting in improved endothelium-dependent relaxation and basal NO release. These data provide further support for the feasibility of using GTPCH gene transfer to correct endothelial dysfunction brought about by BH4 deficiency.

By assessing the amount of hemagglutinin-tagged GTPCH protein that results from AdGTPCH transduction, we can correlate the level of GTPCH activity brought about by gene transfer with the level of NO production. When viral transduction resulted in high expression of the GTPCH gene, the vessel relaxed more than when GTPCH gene transfer was less efficient, providing further evidence that increased GTPCH expression in transduced vessels is responsible for increasing NO production. Increased NO synthesis was made possible by the GTPCH-induced increase in BH4 availability.

A transgenic mouse model in which human GTPCH overexpression was targeted to endothelial cells under the control of the Tie2 promotor exhibited increased BH4 levels in vascular tissues in vivo and increased NOS activity (19). When these transgenic mice were made diabetic by streptozotocin injection, they maintained sufficient BH4 levels to support normal endothelial vasodilatory responses to acetylcholine. This contrasted with the wild type diabetic mice, which exhibited decreased BH4 levels and deficient NO-mediated endothelial function. Thus, a method for increasing BH4 concentrations in endothelial cells exhibiting impaired NO synthesis may serve as a means of alleviating endothelial dysfunction and increasing NO biosynthesis in diabetes and other diseases associated with endothelial dysfunction and decreased NO bioavailability.

In addition to decreased de novo BH4 synthesis, increased oxidation of BH4 to dihydrobiopterin may occur and has been shown to decrease BH4 levels in diabetes (19). When BH4 levels fall, eNOS is “uncoupled” from its normal substrate. Electrons are transferred to molecular oxygen rather than arginine, producing superoxide (20). Superoxide may interact directly with BH4 to oxidize it. Alternatively, superoxide can react with NO to form peroxynitrite, which oxidizes BH4 with even greater efficiency. Loss of BH4, then, leads to further oxidative injury and sustained endothelial dysfunction. Zheng et al. (18) demonstrated that GTPCH gene transfer could reduce superoxide formation noted in the DOCA-salt rat, reversing the BH4 deficiency and endothelial dysfunction noted in this disease model and improving endothelium-dependent relaxation.

While short-term treatment with exogenous BH4 has been shown to improve endothelial cell function and vessel reactivity (11-13), chronic in vivo pharmacological administration is not a practical solution. Exogenous BH4 is easily oxidized to dihydrobiopterin, which no longer functions as an eNOS cofactor and can actually compete with BH4 for eNOS binding (21). Harding et al. (22) reported that BH4 injected intravenously was rapidly cleared from the circulation and taken up by the liver and kidney, with uptake into skeletal muscle being relatively low. The half-life of BH4 was determined to be only 30 minutes, necessitating repeated injections to maintain muscle BH4 content at levels sufficient to support BH4-dependent enzyme activity.

Sepiapterin has been shown to function in vitro to restore BH4 levels in endothelial cells (6). Sepiapterin reduced postischemic injury in the rat heart brought about by myocardial stunning and infarction apparently by ameliorating NO availability, thereby attenuating neutrophil activation in ischemia/reperfusion (23). However, the suitability of sepiapterin as an in vivo treatment in humans has not been proven. Indeed, BH4 supplementation in hypercholesterolemic rabbits brought about via high-dose sepiapterin actually worsened NO-mediated endothelial function, possibly due to uncoupling of eNOS as a result of competition with BH4 at the active site of the enzyme (24).

Thus, the inventor has shown that GTPCH gene transfer can significantly increase BH4 levels and NO production in coronary endothelial cells as well as increase NO-mediated dilation in isolated vascular segments from type I and type II diabetic rats. While GTPCH gene transfer did result in an equivalent increase in BH4 levels in vascular smooth muscle cells, this was not accompanied by an increase in NO synthesis, presumably due to lack of eNOS in smooth muscle cells. Therefore GTPCH gene transfer provides a specific means of increasing endogenous levels of BH4 for preservation of endothelial function in diabetes.

Interestingly, GTPCH gene transfer appears most beneficial in those conditions where BH4 deficiency exists. Zheng et al. (18) demonstrated that GTPCH gene transfer restores the levels of BH4 necessary for basal NO production and normal vasoreactivity in hypertensive rats exhibiting a BH4 deficiency. In contrast, Hynes et al. (25) demonstrated increased BH4 and GTPCH activity in carotid arteries following GTPCH gene transfer in normal rabbits, yet observed no increase in endothelium-dependent relaxation in response to acetylcholine. It may be that increasing levels of BH4 beyond a supposedly “normal” level does not confer any additional benefit. Indeed, Cai et al. (7) showed only a small increase in NO production following GTPCH gene transfer in normal human dermal microvascular cells, despite a large increase in BH4 levels. These observations provide support for overexpression of GTPCH as a therapeutic strategy for amelioration of the endothelial BH4 deficiency in diabetes and as a powerful and specific means of retarding or reversing progression of vascular disease in diabetic individuals. These studies lay the groundwork for gene therapy directed at GTPCH that includes the compositions, systems and methods of the present invention for preventing and/or treating the vascular complications of diabetes and other diseases associated with endothelial dysfunction and decreased NO bioavailability.

### Example 2

Design and Use of a Nanoparticle to Improve Endothelial Cell Function

A targeting nanoparticle complex may be prepared containing a condensed DNA core (containing a nucleic acid sequence encoding GTPCH), a hydrophobic polymer layer, a hydrophilic polymer layer, and a layer of ligand at the outer surface (Lox-1 receptor monoclonal antibody). These nanoparticles are designed to deliver the nucleic acid sequence to damaged endothelial cells, increasing tetrahydrobiopterin and nitric oxide synthesis in these dysfunctional cells. By increasing production of GTPCH protein, and thus tetrahydrobiopterin and nitric oxide synthesis in cells, the impairment in the cell can be reversed or reduced, and endothelial function will be improved. In addition, by increasing the cellular level of tetrahydrobiopterin in the cell, the antioxidant pool is increased and the cell will be protected from future oxidative damage.

### Example 3

Assembly of a Nanoparticle Complex

The assembled nanoparticle complex can have a layered structure with a condensed DNA core, a hydrophobic polymer layer, a hydrophilic polymer layer and a layer of ligand at the outer surface. This layered nanoparticle will be generated by the self assembly of DNA on mixing with a tripartite polymer conjugate. This tripartite polymer conjugate will include a DNA binding cationic polymer such as polyethyleneimine (PEI), a hydrophilic polymer polyethylene glycol (PEG) covalently conjugated to primary amines of PEI, and a ligand (e.g., Lox-1 monoclonal antibody [Lox-1-mAb]) conjugated to the distal end of PEG. On mixing with DNA, PEI will bind and condense the plasmid DNA forming a hydrophobic core and the hydrophilic PEG polymer will form a layer around the core providing a steric protective coat. Since the ligand is attached to the end of PEG, it will be exposed on the surface of the resulting nanoparticle (50-100 nm in size). While the steric layer provides protection from non-specific interactions involving serum proteins and non-target cells, exposed ligand provides targeted delivery. If the ligand interferes with the self assembly process due to its large size, the synthesis strategy can be modified to conjugate the ligand after the formation of the nanoparticle.

### Example 4

Selective Delivery of Nucleic Acids

To establish that the Lox-1-mAb-coupled nanoparticle (NP) will selectively deliver nucleic acids to Lox-1-expressing endothelial cells, a Lox-1-mAb-NP containing the plasmid for green fluorescent protein (GFP) (Lox-1-mAb-NP-GFP) will be exposed to coronary endothelial cells isolated from non-diabetic rats. Half of the cells will be pre-incubated with 50 μg/ml oxidized LDL to induce Lox-1 expression while half will be pre-incubated in vehicle solution. These Lox-1⁺ and Lox-1⁻ cells, respectively, will be exposed to the Lox-1-mAb-NP-GFP for 6 hours, washed with phosphate buffered saline, and grown in complete growth medium. After 24 hours, cells will be counterstained with 4′,6′-diamindino-2-phenylindole and fixed. The GFP-expressing cells will be enumerated by counting random microscopic fields using a fluorescence microscope.

### Example 5

Selective Delivery of Nucleic Acids in Vessels

To demonstrate the selective delivery of genes to Lox-1-expressing endothelial cells in vessels, the Lox-1-mAb-coupled nanoparticles complexed with the luciferase gene will be injected via the tail vein into diabetic BB (BBd) and Zucker diabetic fatty (ZDF) rats (30 days post onset of diabetes). After 24 hours, vessel segments, tissues, and endothelial cells will be excised for measurement of luciferase activity. Selectivity will be established by the ability of a 20-fold molar excess of soluble Lox-1 mAb to block luciferase activity.

### Example 6

Determination of Beneficial Effects of GTPCH Production

To validate the beneficial effects of GTPCH on endothelial cell function, the Lox-1-mAb-coupled nanoparticles will be constructed with the GTPCH plasmid and injected into rats. After 24 and 72 hours, coronary endothelial cells will be isolated and analyzed for GTPCH expression/activity, tetrahydrobiopterin levels, and nitric oxide synthesis (Meininger C. J., et al. Biochem. J. 349: 353-356, 2000; Wu, G. and Meininger, C. J. Am. J. Physiol. 269: H1312-H1318, 1995; Li, H., et al. J. Chromatogr. B. 746: 199-207, 2000).

The protective effects of increased tetrahydrobiopterin levels (i.e., increased antioxidant status) will be validated by demonstrating that coronary endothelial cells which have taken up the Lox-1-mAb-coupled nanoparticles containing GTPCH exhibit decreased generation of reactive oxygen species and/or reduced induction of Lox-1 following incubation with oxidized LDL. To verify the beneficial effects of GTPCH on vessel reactivity, thoracic aortic segments will be excised from the same rats, cut into rings 3-4 mm in length, mounted on stainless steel wires attached to a force transducer and a micrometer in an organ bath, stretched to the optimum of the length-active tension relationship, pre-constricted with norepinephrine (to approximately half-maximal contraction), and exposed to increasing concentrations of acetylcholine (10⁻¹⁰-10⁻⁴ M) to assess endothelial nitric oxide synthesis.

### Example 7

Generation of Lentiviral Construct Containing GTPCH Nucleic Acid

A Lox-1-mAb-coupled nanoparticle containing a GTPCH lentiviral construct will be prepared. Construction of this vector will be important for establishing long-term expression of the GTPCH sequence in endothelial cells of diabetic animals. Coronary endothelial cells and aortic segments will be removed 24 hours, 72 hours, 1 week, 2 weeks, 4 weeks, 8 weeks and 12 weeks after nanoparticle injection and analyzed as described above.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

1. De Vriese A. S., Verbeuren T. J., Van de Voorde J., Lameire N. H., and Vanhoutte, P. M. (2000) Endothelial dysfunction in diabetes. Br J Pharmacol 130, 963-974.
2. Wu G. and Meininger C. J. (1995) Impaired arginine metabolism and NO synthesis in coronary endothelial cells of the spontaneously diabetic BB rat. Am. J. Physiol. 269, H1312-H1318.
3. Johnstone M. T., Creager, S. J., Scales, K. M., Cusco, J. A., Lee, B. K., and Creager, M. A. (1993) Impaired endothelium-dependent vasodilation in patients with insulin-dependent diabetes mellitus. Circulation 88, 2510-2516.
4. Chan N. N., Vallance P., and Colhoun H. M. (2000) Nitric oxide and vascular responses in type I diabetes. Diabetologia 43,137-147.
5. Meininger C. J., Marinos R. S., Hatakeyama K, Martinez-Zaguilan R., Rojas J. D., Kelly K. A., and Wu G. (2000) Impaired nitric oxide production in coronary endothelial cells of the spontaneously diabetic BB rat is due to tetrahydrobiopterin deficiency. Biochem. J. 349, 353-356.
6. Marinos R. S., Zhang W., Wu G., Kelly K. A., and Meininger, C. J. (2001) Tetrahydrobiopterin levels regulate endothelial cell proliferation. Am. J. Physiol. 281, H482-H489.
7. Cai S., Alp N. J., McDonald D., Smith I., Kay J., Canevari L, Heales S., and Channon K. M. (2002) GTP cyclohydrolase I gene transfer augments intracellular tetrahydrobiopterin in human endothelial cells: effects on nitric oxide synthase activity, protein levels and dimerization. Cardiovasc. Res. 55, 838-849.
8. Fukushima T. and Nixon J. C. (1980) Analysis of reduced forms of biopterin in biological tissues and fluids. Anal. Biochem. 102, 175-188.
9. Li H., Meininger C. J., and Wu G. (2000) Rapid determination of nitrite by reversed-phase high performance liquid chromatography with fluorescence detection. J. Chromatogr. B 746, 199-207.
10. Griffin K. L., Laughlin M. H., and Parker J. L. (1997) Exercise training improves endothelium-mediated vasorelaxation after chronic coronary occlusion. J. Appl. Physiol. 87, 1948-1956.
11. Pieper G. M. (1997) Acute amelioration of diabetic endothelial dysfunction with a derivative of the nitric oxide synthase cofactor, tetrahydrobiopterin. J. Cardiovasc. Pharmacol. 29, 8-15.
12. Pannirselvam M., Verma S., Anderson T. J., and Triggle C. R. (2002) Cellular basis of endothelial dysfunction in small mesenteric arteries from spontaneously diabetic (db/db −/−) mice: role of decreased tetrahydrobiopterin bioavailability. Br. J. Pharmacol. 136, 25-263.
13. Heitzer T., Krohn K., Albers S., and Meinertz, T. (2000) Tetrahydrobiopterin improves endothelium-dependent vasodilation by increasing nitric oxide activity in patients with type II diabetes mellitus. Diabetologia 43, 1435-1438.
14. Pieper G. M., Moore-Hilton G., and Roza A. M. (1996) Evaluation of the mechanism of endothelial dysfunction in the genetically-diabetic BB rat. Life Sci. 58, 147-152.
15. Kohli R., Meininger C. J., Haynes T. E., Yan W., Self J. T. and Wu G. (2004) Dietary L-arginine supplementation enhances endothelial nitric oxide synthesis in streptozotocin-induced diabetic rats. J. Nutrition 134, 600-608.
16. Etgen G. J. and Oldham B. A. (2000) Profiling of Zucker diabetic fatty rats in their progression to the overt diabetic state. Metabolism 49, 684-688.
17. Mitchell B. M., Dorrance A. N. and Webb, R. C. (2003) GTP cyclohydrolasel inhibition attenuates vasodilation and increases blood pressure in rats. Am. J. Physiol. 285, H2165-H2170.
18. Zheng J-S., Yang X-Q., Lookingland K. J., Fink G. D., Hesslinger C., Kapatos G., Kovesdi I. and Chen, A. F. (2003) Gene transfer of human guanosine 5′-triphosphate cyclohydrolase I restores vascular tetrahydrobiopterin level and endothelial function in low renin hypertension. Circulation 108, 1238-1245.
19. Alp N. J., Mussa S., Khoo J., Cai S., Guzik T., Jefferson A., Goh N., Rockett, K. A. and Channon K. M. (2003) Tetrahydrobiopterin-dependent preservation of nitric oxide-mediated endothelial function in diabetes by targeted transgenic GTP-cyclohydrolase I overexpression. J. Clin. Invest. 112, 725-735.
20. Vasquez-Vivar J., Kalyanaraman B., Martasek P., Hogg N., Masters B. S. S., Karoi H., Tordo P and Pritchard, K. A. Jr. (1998) Superoxide generation by endothelial nitric oxide synthase: The influence of cofactors. Proc. Natl. Acad. Sci. USA 95, 9220-9225.
21. Vasquez-Vivar J., Martasek P., Whitsett J., Joseph J. and Kalyanaraman B. (2002) The ratio between tetrahydrobiopterin and oxidized tetrahydrobiopterin analogues controls superoxide release from endothelial nitric oxide synthase: an EPR spin trapping study Biochem. J. 362, 733-739.
22. Harding C. O., Neff M., Wild K., Jones K., Elzaouk L., Thony B. and Milstien S. (2004) The fate of intravenously administered tetrahydrobiopterin and its implications for heterologous gene therapy of phenylketonuria. Molec. Genet. Metab. 81, 52-57.
23. Tiefenbacher C. P., Lee C-H., Kapitza J., Dietz V. and Niroomand F. (2003) Sepiapterin reduces postischemic injury in the rat heart. Pflugers Arch.—Eur. J. Physiol. 447, 1-7.
24. Vasquez-Vivar J., Dugquaine D., Whitsett J., Kalyanaraman B. and Rajagopalan S. (2002) Altered tetrahydrobiopterin metabolism in atherosclerosis: implications for use of oxidized tetrahydrobiopterin analogues and thiol antioxidants. Arterioscler. Thromb. Vasc. Biol. 22, 1655-1661.
25. Hynes, S. O., Smith L. A., Richardson D. M., Kovesdi I., O'Brien T. and Katusic Z. S. (2004) In vivo expression and function of recombinant GTP cyclohydrolase I in the rat carotid artery. Am. J. Physiol. 286, H570-574.

1. A method for delivering nucleic acid to cells of a recipient subject, comprising the steps of:
contacting Lox-1-expressing endothelial cells with a delivery system, the delivery system comprising: a ligand associated with a nanoparticle carrier, wherein said ligand is capable of binding to Lox-1-expressing endothelial cells and an isolated and purified nucleic acid associated with the carrier encoding a GTP-cyclohydrolase I; and
administering the delivery system to the recipient subject under conditions such that at least a portion of the Lox-1-expressing endothelial cells are contacted by the delivery system.

2. The method of claim 1, wherein the cells are part of vascular tissue.

3. The method of claim 1, wherein the Lox-1-expressing endothelial cells are cells damaged by or reactive to a cardiovascular disease.

4. The method of claim 1, wherein the nucleic acid is contained within an expression vector, the vector comprising a promoter sequence operably linked to the nucleic acid.

5. The method of claim 1, wherein the nucleic acid comprises an expression vector comprising a promoter sequence operably linked to the nucleic acid and the promoter sequence is a viral promoter sequence.

6. The method of claim 1, wherein the ligand comprises an antibody reactive with Lox-1.

7. The method of claim 1, wherein the ligand comprises an antigen binding portion of an antibody.

8. The method of claim 1, wherein the ligand comprises an antigen binding portion of a monoclonal antibody.

9. The method of claim 1, wherein the ligand comprises a peptide.

10. The method of claim 1, wherein the carrier comprises a polymer.

11. The method of claim 1, wherein the carrier comprises a liposome.

12. The method of claim 1, wherein the administering comprises intravenous injection.

13. The method of claim 1, wherein the recipient subject is a human.

14. The method of claim 1, further comprising following the administering testing the recipient subject for evidence of increased nitric oxide synthesis.

15. A delivery system, the system comprising: a Lox-1 binding agent and a nucleic acid encoding a GTP-cyclohydrolase I associated with a nanoparticle carrier, wherein the Lox-1 binding agent targets the delivery system to Lox-1 expressing cells for delivery of the nucleic acid.

16. The system of claim 15, wherein the carrier comprises a polymer.

17. The system of claim 15, wherein the nanoparticle comprises a core and a polymeric surface, wherein the nucleic acid is associated with the core and the ligand is associated with the surface.

18. The system of claim 15, wherein the nanoparticle comprises a biocompatible polymer.

19. The system of claim 15, wherein the nanoparticle comprises a biodegradable polymer.

20. A nanoparticle comprising a ligand capable of binding to Lox-1.
